(19) United States
(12) Patent Application Publication
(10) Pub. No.: US 2024/0051907 A1
(43) Pub. Date: Feb. 15, 2024

(54) CATALYST SYSTEM

(71) Applicant: SYMRISE AG, Holzminden (DE)

(72) Inventors: Angela KÖCKRITZ, Berlin (DE); Elka KRALEVA, Rostock (DE); Katja NEUBAUER, Rostock (DE); Regina BIENERT, Rostock (DE); Stefan LAMBRECHT, Hehlen (DE); Johannes PANTEN, Höxter (DE); Michael SEBEK, Rostock (DE); Bernhard RUSSBÜLDT, Höxter (DE)

(21) Appl. No.: 18/267,563

(22) PCT Filed: Jan. 22, 2021
(86) PCT No.: PCT/EP2021/051455

§ 371 Date: Jun. 15, 2023

(30) Foreign Application Priority Data
Jan. 27, 2020 (WO) PCT/EP2020/051924

(51) Int. Cl. C07C 45/29; B01J 31/04; B01J 31/02; B01J 31/22; C07C 29/56

(52) U.S. Cl. CPC C07C 45/29; B01J 31/04; B01J 31/0238; B01J 31/0271; B01J 31/2243; B01J 31/0208; C07C 29/56; B01J 2231/763; B01J 2531/845; B01J 2531/26; B01J 2231/52; C07C 2601/16

(57) ABSTRACT

Proposed is a catalyst system specifically for the rearrangement of epoxides into allyl alcohols comprising or consisting of (a) a salt of formula (I) XY (I) in which X represents Zn²⁺ and/or Co²⁺ and Y represents an anion selected from the group formed from laurate, palmitate, stearate, picolinate, glycinate, gluconate, naphthenate, 2-hexyldecanoate, 2-octyldodecanoate, cyclohexane butyrate and mixtures thereof, and (b) at least one aminophenol.

### FIELD OF THE INVENTION

The invention is in the field of heterogeneous catalysis and relates to catalysts for the rearrangement of epoxides and corresponding methods for the preparation of allylic alcohols and alpha,beta-unsaturated carbonyl compounds using these catalysts.

### TECHNOLOGICAL BACKGROUND

Allylic alcohols are widely used in the chemical industry, for example, as flavour and fragrance ingredients. Moreover, they may serve as intermediate products, for example, in the manufacture of alpha,beta-unsaturated carbonyl compounds by Oppenauer oxidation. In general, the rearrangement of epoxides to allylic alcohols and further oxidation of obtained alcohols may be expressed as follows:

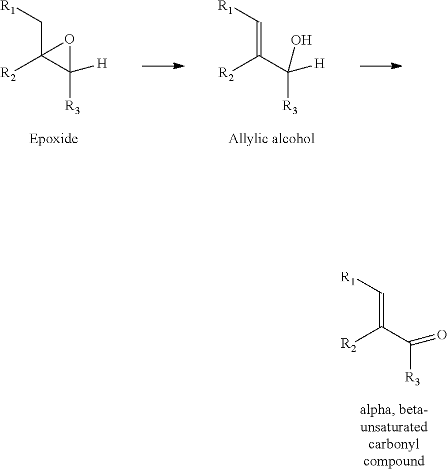

where R1, R2, and R3 represent a hydrogen atom, alkyl, aryl, aralkyl groups, or together form a cycloalkyl group.

Traditional methods used for the rearrangement of epoxides to allylic alcohols typically include the use of at least a stoichiometric amount, or, in most reactions, a large excess of expensive reagents, such as, for example, lithium amide, lithium diisopropylamide, butyl lithium, aluminium amide, potassium butoxide. In addition, when the opening of the epoxide ring proceeds in various directions, these methods lack the selectivity and flexibility to lead the process to the formation of a specific product [cf. J K Crandall and M. Apparu: “Base-promoted Isomerizations of Epoxides”, in: Organic Reactions, John Wiley and Sons, Inc., Vol. 29, pp 345-443 (1983).]

The following Scheme 1 exemplarily shows the various products that may form during the rearrangement of 1,2-Limonene oxide:

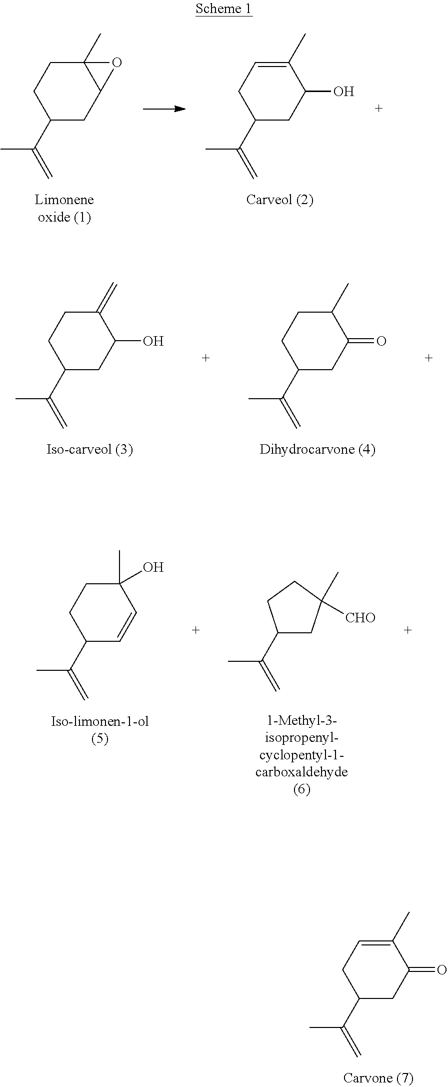

It is generally accepted that proton abstraction in the rearrangement of epoxides to allylic alcohols occurs at the least substituted carbon (J. Gorzynsky Smith: Synthetically Useful Reactions of Epoxides. Synthesis, 1984, (8), pp. 629-656). According to this rule in the rearrangement of 1,2-Limonene oxide (see the scheme above) promoted by strong bases a preferential formation of iso-carveol (3) occurs (Y. Bessiere and R. Derguini-Boumechal, J. Chem. Res. (S), 1977, (12), pp. 304-305). The highest selectivity to carveol (2), which is a flavour component and an intermediate in synthesis of carvone (7), was 22%.

As may be seen, traditional methods for epoxide rearrangement to allylic alcohols fail to selectively produce carveol from 1,2-Limonene oxide. It was reported that in some cases the manufacture of allylic alcohols from epoxides is accompanied by formation of a significant amount of the corresponding unsaturated carbonyl compound. For example, in the rearrangement of 1,2-Limonene oxide over metal oxides and binary oxides, the selectivity of carvone (7) formation was as high as 35% at 75% conversion of epoxide. However, total selectivity to carveol and carvone was only 59% (J. Jayasree, Ind. J. Chem., 1997, Vol. 36A, (9), pp. 765-768).

Formation of unsaturated carbonyl compounds during the rearrangement of epoxides results by Oppenauer oxidation of the allylic alcohol. This reaction is possible because some epoxide rearrangement catalysts are also capable of catalysing the Oppenauer oxidation, and by-products of the rearrangement—dihydrocarvone (4) and aldehyde (6) in the discussed example—may act as hydrogen acceptors.

It is clear that the more alpha,beta-unsaturated compound is produced, the lower is total yield of allylic alcohol and alpha,beta-unsaturated carbonyl compound, since more epoxide undergoes undesired transformation to the corresponding carbonyl compounds (4 or 6), and further saturated alcohols (8) or (9). The sequence of the epoxide rearrangement and the Oppenauer oxidation of allylic alcohol utilising carbonyl by-products as hydrogen acceptors is presented in Scheme 2.

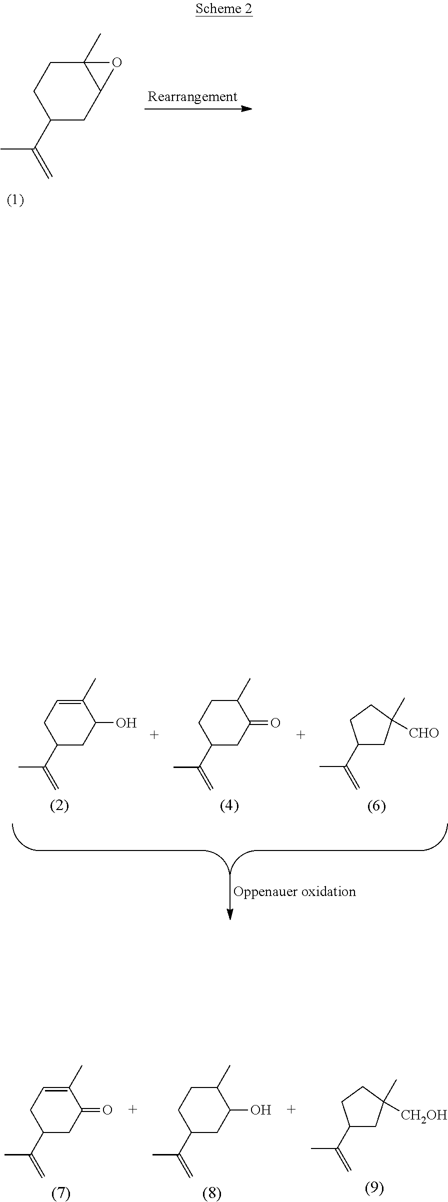

### RELEVANT STATE OF THE ART

The most frequently used catalysts for rearranging allylic alcohols are aluminium isopropoxide (E H Eschinasi, Isr. J. Chem., 1968, 6, pp. 713-721), titanium alkoxide (JP 50 058031 A, and zirconium butoxide (U.S. Pat. No. 4,496,776). In the presence of these catalysts, the selectivity of the 1,2-Limonene oxide rearrangement for carveol is between 24% (aluminium isopropoxide) and 60% (titanium isobutoxide). Shared disadvantages of these catalysts are the complicated processing of the reaction mixtures and a low activity and selectivity, which restricts their applications.

Numerous heterogeneous catalysts have also been suggested for the rearrangement of epoxides to allylic alcohols. They include metal oxides, particularly different grades of aluminium acid molecules, silicon dioxide, titanium dioxide, zirconium oxide, and mixed oxides (see review by K. Tanabe, R. Ohnishi, K. Arata: “Rearrangement of epoxides over solid acid and base catalysts”, chapter 2.5, in: Terpene Chemistry, ed. J. Varghese. Tata McGraw-Hill Publishing Company, Ltd., 1982, pp. 67-88). The highest selectivity achieved in the 1,2-Limonene oxide rearrangement to carveol catalysed by metal oxides was 59% over aluminium oxide (K. Arata, K. Tanabe, Chem. Letters, 1976, pp. 321-322).

From the two U.S. Pat. Nos. 2,426,624 and 2,986,585, methods for the production of allylic alcohol based on a propylene oxide rearrangement in the presence of lithium phosphate are known. The methods are carried out at a temperature of from 275 to 300° C. Selectivity of the allylic alcohol formation is about 80%.

Silica-supported lithium phosphate (U.S. Pat. No. 5,455,215) and zirconium oxide-supported sodium phosphate (JP 11 049709 A) have also been used to effect the rearrangement of epoxides.

Rearrangement of 1,2-Limonene oxide in the presence of lithium phosphate was studied by S. G. Traynor et al. (Proceedings of the VIII^{th} International Congress of Essential Oils. Fedarom, Grasse, 1980. pp. 591-594). Reaction was very slow. Selectivity of trans-1,2-Limonene oxide conversion to cis-carveol was 18.1% with a conversion of 66.2% (57 hours, 200° C.). Selectivity of cis-1,2-Limonene oxide conversion to trans-carveol was 13.6% with a conversion of 69.9% (57 hours, 200° C.). During this reaction, significant amounts of carbonyl compounds—aldehyde (6) and ketone (4)—were formed (10.9% and 4.3%). The main product was iso-carveol (3)—68.7 percent in the case of a cis-1,2-Limonene oxide rearrangement, and 59.9 percent in the case of a trans-1,2-Limonene oxide rearrangement.

Finally, from EP 1404635 B1 (MILENNIUM SPECIALITY) a catalyst system is known which is suitable both for the rearrangement of epoxides to allylic alcohols and for an Oppenauer oxidation, disclosing, for example, the combination of zinc octanoate and aminophenol. Therein, however, selectivity to carveol is below 40%.

### OBJECT OF THE INVENTION

In many instances, the preparation of alpha,beta-unsaturated carbonyl compounds from epoxides by the sequence of rearrangement and oxidation reactions is an ultimate goal. However, none of the traditional techniques is capable of combining these two steps to produce high yields of alpha,beta-unsaturated carbonyl compound, particularly in a one-pot process.

A first object of the present invention was, therefore, to provide catalysts by means of which epoxides, particularly 1,2-Limonene epoxide, are converted to allylic alcohols with conversions of at least 70%, and, preferably, of at least 80%, and allowing to obtain yields of at least 40%, preferably, at least 45%.

### DESCRIPTION OF THE INVENTION

A first subject matter of the invention relates to a catalyst system, particularly for the rearrangement of epoxides to allylic alcohols, and for the production of alpha,beta-unsaturated carbonyl compounds, comprising or consisting of
(a) a salt of formula (I)

XY (I)
Where X represents Zn²⁺ and/or Co²⁺ and Y represents an anion which is selected from the group consisting of laurate, palmitate, stearate, picolinate, glycinate, gluconate, naphthenate, 2-hexyldecanoate, 2-octyldodecanoate, cyclohexane butyrate and the mixtures thereof, and
(b) at least one aminophenol.

Surprisingly, it was found that the catalyst system, particularly pre-formed catalysts, catalysed both the rearrangement of epoxides to allylic alcohols and also the Oppenauer oxidation of allylic alcohols to the corresponding alpha,beta-unsaturated carbonyl compounds, which was carried out at conversions of above 70% and with yields of more than 40%.

### Catalyst System

The catalyst system according to the invention consists of two components, i.e., (a) primary catalyst and (b) an activator or modifier. Component (a) is a defined salt of divalent zinc or cobalt, particularly, zinc stearate, cobalt stearate, zinc glycinate, zinc naphthenate, and mixtures thereof. Herein, the term “salt” is understood to mean that it is an at least mainly ionic compound containing Zn²⁺ and/or Co²⁺ cations as well as a corresponding stoichiometric number of said anions, so that a neutral compound is present.

Component (b) has the task of activating component (a), as component (a) alone very often does not have any catalytic properties, or just very few. At the same time, being a modifier, it has the property to control selectivity. Suitable activators/modifiers are aminophenols, particularly, 2-Aminophenol.

Particularly preferred are catalyst systems which are a combination of (a) zinc stearate, cobalt stearate, zinc glycinate, zinc-2-ethyl hexanoate, zinc naphthenate, and mixtures thereof with (b) 2-Aminophenol.

The catalyst systems may include components (a) and (b) in a weight ratio of from about 10.000:1 to about 10:1, preferably, from about 5.000:1 to about 100:1, and particularly, from 1.000:1 to about 500:1.

### Method

A second subject matter of the invention relates to a method for the rearrangement of epoxides to allylic alcohols, comprising or consisting of the following steps:
(a) Providing a starting compound having at least one epoxide group;
(b) Providing a catalyst system as described above;
(c) Adding the catalyst system to the starting compound and heating the mixture to temperatures within the range of from about 155 to about 250° C. for a period of from about 1 to about 10 hours;
(d) Allowing the mixture to cool, and, optionally, separation of the one or more allylic alcohols formed from the residue.

A third subject matter of the invention relates to a method for the rearrangement and Oppenauer oxidation of epoxides to alpha,beta-unsaturated carbonyl compounds, comprising or consisting of the following steps:
(a) Providing a starting compound having at least one epoxide group;
(b) Providing a catalyst system as described above;
(c) Providing a consumption agent;
(d) Adding the catalyst system to the starting compound and the consumption ketone, and heating the mixture to temperatures within the range of from about 170 to about 250° C. for a period of from about 1 to about 10 hours;
(e) Allowing the mixture to cool, and, optionally, separation of the one or more alpha-beta-unsaturated carbonyl compounds formed from the residue.

According to the present invention, a broad spectrum of epoxides may be converted to allylic alcohols. Examples of terminal, cyclic, di-substituted, tri-substituted epoxides are 1,2-Limonene oxide, 8,9-Limonene oxide, alpha-Pinene oxide, beta-Pinene oxide, 2,3-Carene oxide, 3,4-Carene oxide, 1,2-Terpinolene oxide, 4,8-Terpinolene oxide, Sylvestrene oxide, 1,2-Menthene oxide, 2,3-Menthene oxide, 3,4-Menthene oxide, 7,8-Dihydromyrcene oxide, Caryophyllene oxide, 1,2-Epoxy cyclododecane, and the like. The rearrangement of epoxides to allylic alcohols according to the present invention may be performed by contacting epoxide with the catalyst system under suitable reaction conditions, e.g., at an increased temperature, usually under reflux. The epoxide rearrangement may be performed batch-wise or continuously. Other suitable steps may also be included into the rearrangement process. For example, it may be preferred to remove water, which is contained in the starting materials, or which has been formed during the process. In these instances, water may be removed before or during the rearrangement by known methods: if an allylic alcohol is a final product, rearrangement may be stopped, e.g., after all epoxide has been reacted, or when the desired conversion has been achieved. Furthermore, the following steps may be applied after rearrangement. For example, catalyst may be removed by any suitable method (filtration, cleaning, extraction, distillation, etc.), and product, e.g., allylic alcohol, may be isolated and purified using a method known in the art, such as, for example, by distillation or crystallisation.

Both methods may be performed at the same or very similar temperatures, particularly within the range of from about 200 to about 230° C., and particularly of from about 210 to about 220° C.

Catalyst component (a) is usually used in amounts of from about 0.05 to about 5 mol %, preferably, about 0.1 to about 1 mol %, and catalyst component (b) in amounts of from about 0.01 to about 0.00001 mol %, preferably, about 0.001 to about 0.0001 mol %, each based on the starting compound.

A further advantage of the invention is that the rearrangement and the Oppenauer oxidation may be performed either simultaneously as a “one-pot reaction”, or subsequently.

Within the context of the Oppenauer oxidation it is reasonable to use a consumption agent. If the reaction is performed as a one-pot reaction where both reactions are proceeding at the same time, it is possible, in principle, to use the consumption agent together with the catalyst system; however, it is more advantageous to firstly allow the reaction to proceed for a certain amount of time so that a sufficient amount of allylic alcohol is present, and to add the consumption agent only later, thus starting the oxidation process.

Suitable consumption agents are, particularly, aldehydes or ketones, including the quinones. Typical examples encompass benzaldehyde, acetone, cyclohexanone, benzoquinone, or isophorone. Typically, these are used in very low amounts, for example, from about 1 to about 100 mmol, preferably, from about 10 to about 50 mmol consumption agent, based on the amount of starting compound.

Further, it has proved to be advantageous to periodically remove the alcohol formed from the consumption agent, which, preferably, represents cyclohexanone, from the reaction mixture, as this way the balance is shifted to the product side.

### EXAMPLES

### General Scheme

The following scheme exemplarily illustrates the rearrangement of 1,2-Limonene epoxide to the corresponding allylic alcohol carveol on the one hand, and its further reaction by Oppenauer oxidation on the other, in order to form the alpha-beta-unsaturated carbonyl compound carvone.

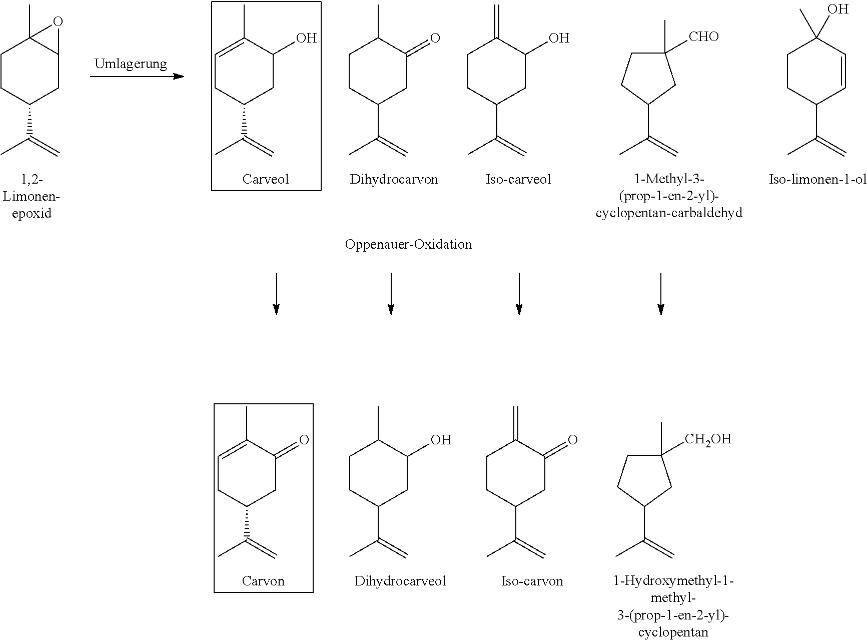

### Examples 1 to 12, Comparison Examples V1 to V25

### Rearrangement of 1,2-Limonene Epoxide to Carveol

16 mmol 1,2-Limonene epoxide (“LE”—cis:trans=58:42 (mol/mol)), 1.3 mol % of catalyst, and 2-Aminophenol (“2AP”) were placed into a three-neck flask equipped with a reflux cooler, an internal thermometre, and a capillary for dosing a gas. The mixture was heated to 205 to 210° C. while stirring, and was further continued to be stirred at this temperature for the time indicated. After cooling down, product composition was determined by means of gas chromatography. Table 1 summarises the results:

TABLE 1
Rearrangement of 1,2-Limonene oxide (conversions and yields in GC %)

| | | | | | Yields | | |
|---|---|---|---|---|---|---|---|
| | | 2AP | | Conversion | Iso- | | |
| Exp. | Catalyst | (mmol) | t(h) | LE | Carveol | Carveol | Carvone |
| V1 | Zn₃(citrate)₂ 2 H₂O | 0.32 | 4 | 22.2 | 0.5 | 1.2 | 0.3 |
| V2 | Cu(stearate)₂ | 0.33 | 4 | 21.8 | 1.7 | 1.9 | 0 |
| V3 | Li-stearate | 0.33 | 4 | 17.1 | 0.9 | 2.3 | 0 |
| V4 | CdO | 0.32 | 4 | 8 | 2.1 | 3.9 | 2 |
| V5 | Zn(gluconate)₂ H₂O | 0.32 | 4 | 8.7 | 4.2 | 4.1 | |
| V6 | Pr(NO₃)₃ 6 H₂O | 0.32 | 4 | 73.4 | 8.4 | 5.4 | 7.1 |
| V7 | Ca(stearate)₂ | 0.32 | 4 | 8.3 | 3 | 5.5 | 1.2 |
| V8 | In(tetramethylheptanedione)₃ | 0.32 | 4 | 19 | 9.3 | 5.7 | 0.6 |
| V9 | Co-naphthenate | 0.32 | 4 | 98.5 | 12.1 | 6.7 | 10.7 |
| V10 | Cd(acetate)₂ | 0.32 | 4 | 36.9 | 8 | 7.2 | 8.4 |
| V11 | Mg-stearate | 0.32 | 4 | 59.4 | 4.3 | 8.3 | 3.2 |
| V12 | In(acetylacetonate)₃ | 0.32 | 4 | 41 | 19.8 | 10.6 | 2.6 |
| V13 | Fe-naphthenate | 0.32 | 4 | 90.3 | 15 | 14.1 | 7.8 |
| V14 | Ga(tetramethylheptanedione)₃ | 0.32 | 4 | 59.4 | 22.8 | 15.4 | 2.3 |
| V15 | NdCl₃ | 0.32 | 4 | 74.5 | 8.9 | 15.4 | 7.5 |
| V16 | Sn(acetylacetonate)₂ | 0.32 | 4 | 70.6 | 31.2 | 15.8 | 2.6 |
| V17 | Zn-orotate 2 H₂O | 0.32 | 4 | 63.2 | 18.1 | 16.1 | 1 |
| V18 | In(acetate)₃ | 0.32 | 4 | 65.5 | 34.9 | 16.9 | 2.6 |
| V19 | Zn(acetylacetonate)₂ | 0.32 | 4 | 81.3 | 9.2 | 26.6 | 8.7 |
| V20 | Ni(acetylacetonate)₂ | 0.32 | 4 | 50.1 | 3.9 | 27 | 4.3 |
| V21 | Zn(octoate)₂ | 0.32 | 3 | 66.1 | 7.5 | 29.2 | 8.4 |
| V22 | Zn(tetramethylheptanedione)₂ | 0.32 | 4 | 52.1 | 7.7 | 30 | 5.7 |
| V23 | Fe(acetylacetonate)₃ | 0.32 | 4 | 84.5 | 15.1 | 37.4 | 4.8 |
| V24 | Zn(tetradecyloctadecanoate)₂ | 0.32 | 4 | 82.4 | 4.1 | 37.5 | 16.1 |
| V25 | Zn(octoate)₂ | 0.32 | 4 | 75.7 | 7.9 | 38.7 | 13.5 |
| 1 | Zn(2-octyldodecanoate)₂ | 0.32 | 4 | 83.7 | 5.3 | 40.0 | 12.6 |
| 2 | Zn(cyclohexane butyrate)₂ | 0.32 | 4 | 82.5 | 12.4 | 40.0 | 17 |
| 3 | Zn(stearate)₂ | 3.00 | 7 | 99.3 | 4.5 | 41.9 | 8.1 |
| 4 | Zn(2-hexyldecanoate)₂ | 0.32 | 4 | 84.7 | 4.5 | 43.3 | 13.7 |
| 5 | Zn(palmitate)₂ | 0.32 | 4 | 81.4 | 8.2 | 45 | 17.8 |
| 6 | Zn(picolinate)₂ | 0.32 | 4 | 99.5 | 13.1 | 48.7 | 12 |
| 7 | Zn(laurinate)₂ | 0.32 | 4 | 73.1 | 8 | 49 | 16.8 |
| 8 | Zn(picolinate)₂ | 0.32 | 4 | 99.7 | 10.4 | 49.7 | 9.9 |
| 9 | Co(stearate)₂ | 0.33 | 4 | 90.6 | 9.2 | 49.8 | 13 |
| 10 | Zn(glycinate)₂ H₂O | 0.32 | 4 | 97.4 | 13.3 | 53.5 | 15.4 |
| 11 | Zn-naphthenate | 0.32 | 4 | 86.2 | 8.8 | 54.7 | 13.9 |
| 12 | Zn(stearate)₂ | 0.32 | 4 | 93.6 | 13.7 | 55.2 | 20.4 |

Examples and comparison examples show that the object of achieving yields of carveol of at least 40% may only be achieved with the catalyst system according to the invention.

### Examples 13 to 19

### Combination of Rearrangement and Oppenauer Oxidation

16 mmol 1,2-Limonene epoxide (cis:trans=58:42 (mol/mol)), half of the amount of catalyst, and the phenol were placed into a three-necked flask equipped with a reflux cooler, an internal thermometre, and a capillary for dosing a gas. While stirring, the amount was heated to 205 to 210° C. with the reflux cooler switched off, and was continued to be stirred at this temperature for the time indicated (e.g., 3/2). After cooling down to 180 to 190° C., the second half of the amount of catalyst and the consumption agent were added and continued to be stirred at this temperature under reflux for the time indicated (e.g., 3/2). After cooling down, product composition was determined by means of gas chromatography. Results are summarised in Table 2.

### Example 20

### Rearrangement/Oppenauer Reaction as a One-Pot Reaction with Intermediate Removal of Cyclohexanol Formed

16 mmol 1,2-Limonene epoxide (cis:trans=58:42 (mol/mol)), half of the amount of catalyst, and the phenol were placed into a three-necked flask equipped with a reflux cooler, an internal thermometre, and a capillary for dosing a gas. While stirring, the mixture was heated to 205 to 210° C., and was continued to be stirred at this temperature for the time indicated (e.g., 2/2/2). After cooling down to 180 to 190° C., the second half of the amount of catalyst and half of the amount of cyclohexanone were added, and were continued to be stirred under reflux for the time indicated (e.g., 2/2/2). Subsequently, the mixture was cooled and distilled over a distilling link cyclohexanol/unreacted cyclohexanone placed onto the flask. Then, the second half of the amount of cyclohexanone was added to the reaction mixture, and the content of the flask was again brought to reflux for the time indicated (e.g., 2/2/2). After cooling down, product composition was determined by means of gas chromatography; results are also shown in Table 2.

TABLE 2
Rearrangement/Oppenauer oxidation of 1,2-Limonene oxide (conversions and yields in GC %)

| | | | | | | Consump- | | | | | |
|---|---|---|---|---|---|---|---|---|---|---|---|
| | | | | | | tion | | | Yields | | |
| | | Catalyst | | Phenol | Consumption | agent | t | Conversion | Iso- | | |
| Exp. | Catalyst | [mol %] | Phenol | [mmol] | agent | [mmol] | [h] | LE | carveol | Carveol | Carvone |
| 13 | Zn(laurate)₂ | 1.3 | 2-Aminophenol | 0.6 | Cyclohexanone | 14.2 | 3/4 | 95 | 4 | 33.1 | 40.1 |
| 14 | Co(stearate)₂ | 1.3 | 2-Aminophenol | 0.32 | Cyclohexanone | 14.2 | 3/3 | 94.4 | 7.6 | 12.4 | 40.6 |
| 15 | Zn(stearate)₂ | 1.3 | 2-Aminophenol | 0.6 | Cyclohexanone | 14.2 | 3.5/4 | 96.7 | 4.2 | 23.2 | 42.4 |
| 16 | Zn(palmitate)₂ | 1.3 | 2-Aminophenol | 0.6 | Cyclohexanone | 14.2 | 3/4 | 94.6 | 4.5 | 18.6 | 43.5 |
| 17 | Zn(stearate)₂ | 1.3 | 2-Aminophenol | 0.6 | Cyclohexanone | 28.4 | 3/2 | 96.4 | 6.3 | 27.8 | 45.4 |
| 18 | Zn(glycinate)₂ H₂O | 1.3 | 2-Aminophenol | 0.6 | Cyclohexanone | 14.2 | 3/4 | 99.3 | 6 | 29 | 47 |
| 19 | Zn-naphthenate | 1.3 | 2-Aminophenol | 0.6 | Cyclohexanone | 14.2 | 3/4 | 97.4 | 4.9 | 18.1 | 56.1 |
| 20 | Zn(stearate)₂ | 1.3 | 2-Aminophenol | 0.6 | Cyclohexanone | 28.4 | 2/2/2 | 87.9 | 5.3 | 38.4 | 43.8 |

### Examples 21 to 31

### Rearrangements Using Pre-Formed Catalysts

cis-Limonene-1,2-epoxide may be rearranged faster than trans-Limonene-1,2-epoxide. The rearrangement of trans-Limonene-1,2-epoxide is facilitated if cis-Limonene-1,2-epoxide is present in the reaction mixture. Therefore, it is advantageous to utilise as the substrate either pure cis-Limonene-1,2-epoxide or a mixture of cis- and trans-Limonene-1,2-epoxides having a high cis-proportion.

Particularly preferred catalysts are also pre-formed Zn(aminophenolate)₂ complexes 1-8. These allow the rearrangement of the epoxide already at milder reaction temperatures of from 155° C. (see Table, examples 2, 4, 5). They could be produced in analogy to a method of production found in literature (H. R. Hoppe, K. Andrä, Z. Chem. 26 (1986)) as follows:

Under protective gas, 2.5 mL ZnEt₂ in dry toluene (16.3%, 3 mmol) were filled into a dried 100 mL Schlenk flask. While stirring and with a slight flow of argon, 0.635 g (6 mmol) 2-Aminophenol was added in doses via a septum at a temperature of 25° C. within 10-15 mins. A colourless solid precipitated, and the formation of ethane was observed. Crystals attached to the flask wall were carefully washed back into the reaction mixture using 5 mL dry toluene. When addition was completed, the Schlenk flask was heated to 88° C. in an oil bath, and was stirred at this temperature for two hours. Subsequently, the hot reaction mixture was filtered, and the solid obtained was treated with dry toluene until reaching colourlessness of the purifying solvent. Then, the crystals were also washed with dry heptane, dried within the oil pump vacuum, and used as catalysts. As an example, 0.78 g (93% yield) were isolated from Zn(2-Aminophenolate)₂. Structures of pre-formed catalysts are shown below (reference numbers correspond to those in Table 3):

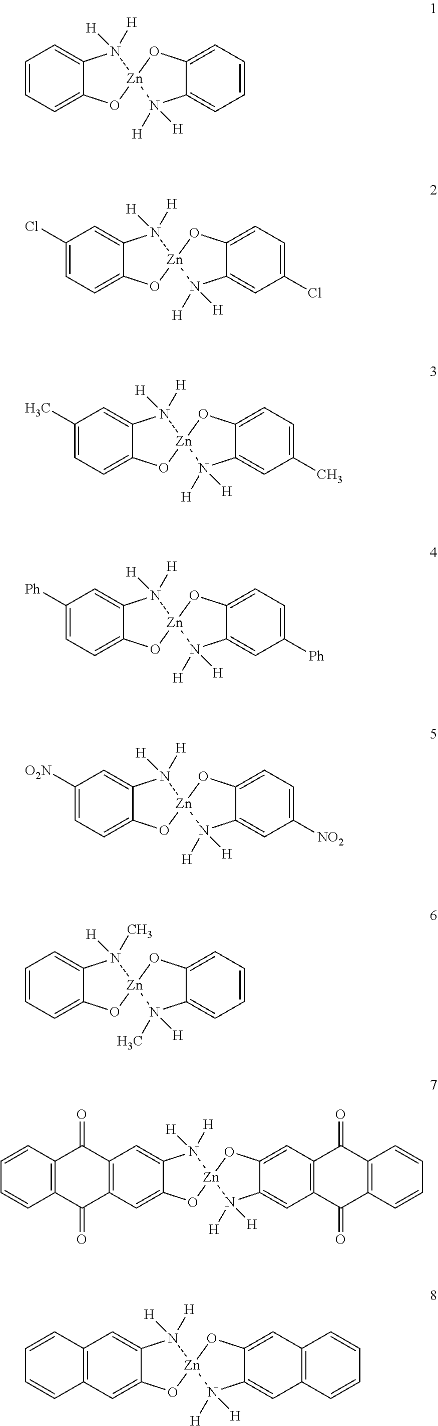

The use of 2-Aminophenols alkylated with cis- or trans-Limonene-1,2-epoxide instead of 2-Aminophenol, in combination with Zn-2-ethylhexanoate also yielded very high yields of carveol plus carvone. With this catalyst system significant differences in reactivity of cis- and trans-(+)-Limonene-1,2-epoxide were determined. Alkylated aminophenols 9-11 were produced as follows:

270 mg (1.77 mmol) cis-(+)-Limonene-1,2-epoxide, 110 mg (0.99 mmol) 2-Aminophenol and 10 mg (0.036 mmol) Zn(2-Aminophenolate)₂ were placed into a 2-necked pear-shaped flask (10 mL) equipped with a reflux cooler and a thermometre. The apparatus was fixed in a preheated oil bath. Internal temperature was 166° C. The reaction mixture was stirred for 2 h 15 mins. After cooling down, products were separated by column chromatography (eluent: ethyl acetate/heptane 3.5:5). Fractions 15-26 (95 mg) contained the monoalkylated product 9. Mono- and dialkylated products 10 and 11 with trans-(+)-Limonene-1,2-epoxide were produced in analogy to this method, separated by column chromatography, and purified. Structures of alkylated aminophenols are given below:

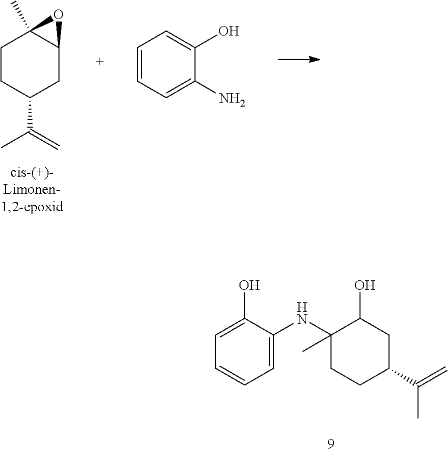 [SMILES: C=C(C)[C@@H]1CC[C@@]2(C)O[C@H]2C1 ; Nc1ccccc1O ; C=C(C)[C@@H]1CCC(C)(Nc2ccccc2O)C(O)C1]

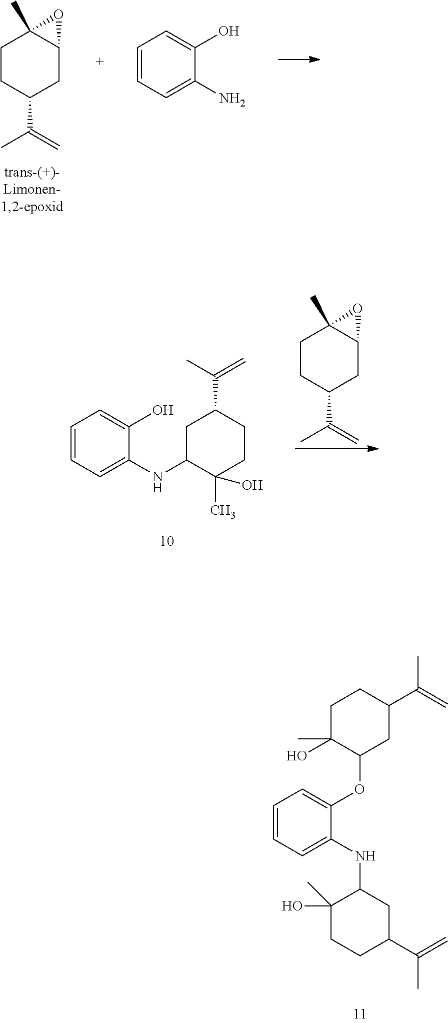 [SMILES: C=C(C)[C@@H]1CC[C@]2(C)O[C@@H]2C1 ; Nc1ccccc1O ; C=C(C)[C@@H]1CCC(C)(O)C(Nc2ccccc2O)C1 ; C=C(C)[C@@H]1CC[C@]2(C)O[C@@H]2C1 ; C=C(C)C1CCC(C)(O)C(Nc2ccccc2OC2CC(C(=C)C)CCC2(C)O)C1]

### Catalytic Reactions

19.7 mmol 1,2-Limonene epoxide (“LE”—cis:trans-=63:37 (mol/mol)) and 0.4 mol % of catalyst were placed into a three-necked flask equipped with a reflux cooler, an internal thermometre, and a capillary for dosing a gas. While stirring, the mixture was heated in an argon atmosphere to the temperature indicated, and was continued to be stirred at this temperature for the time indicated. After cooling down, product composition was determined by means of gas chromatography. Table 1 summarises the results:

TABLE 3
Rearrangement of 1,2-Limonene epoxide with
pre-formed zinc catalysts

| | | | | conver- | Yields [%] | | |
|---|---|---|---|---|---|---|---|
| | | | | sion | Isocarveol + | | |
| | | T | | LE | Dihydro- | | |
| Exp. | Cat. | [° C.] | t [h] | [%] | carvone | Carveol | Carvone |
| 21 | 1 | 200-224 | 0.5 | 98.8 | 3.6 | 80.2 | 7.2 |
| 22 | 1 | 155 | 6 | 79.3 | 2.1 | 68.9 | 1.2 |
| 23 | 2 | 200-224 | 0.5 | 99.7 | 4.6 | 70.3 | 12.2 |
| 24 | 2 | 160 | 3 | 76.2 | 3.0 | 67.2 | 2.1 |
| 25 | 2 | 186 | 6 | 99.0 | 3.6 | 79.3 | 7.8 |
| 26 | 3 | 200-224 | 2 | 98.5 | 4.0 | 77.0 | 7.7 |
| 27 | 4 | 200-224 | 0.5 | 99.1 | 3.8 | 77.3 | 7.9 |
| | | | 3 | 99.7 | 4.3 | 64.4 | 20.7 |
| 28 | 5 | 200-224 | 3 | 96.0 | 5.4 | 57.4 | 18.1 |
| 29 | 6 | 200-224 | 2.5 | 98.7 | 5.6 | 76.2 | 9.6 |
| 30 | 7 | 200-224 | 1,5 | 99.6 | 6.1 | 43.3 | 29.7 |
| | | | 4.5 | 100 | 5.0 | 17.8 | 46.4 |
| 31 | 8 | 200-224 | 0.25 | 92.2 | 6.0 | 68.4 | 5.3 |
| | | | 3 | 100 | 5.9 | 32.3 | 40.7 |

19.7 mmol (+)-Limonene-1,2-epoxide, the indicated amount of Zn-2-Ethylhexanoate, and the indicated amount of aminophenol were placed into a three-necked flask equipped with a reflux cooler, an internal thermometre, and a capillary for dosing a gas. While stirring, the mixture was heated to 200 to 224° C. in an argon atmosphere, and was continued to be stirred at this temperature for the time indicated. After cooling down, product composition was determined by means of gas chromatography. Table 1 summarises the results.

1. A catalyst system, comprising or consisting of
(a) a salt of formula (I)
XY (I)
wherein X represents Zn²⁺ and/or Co²⁺ and Y represents an anion which is selected from the group consisting of laurate, palmitate, stearate, picolinate, glycinate, gluconate, naphthenate, 2-hexyldecanoate, 2-octyldodecanoate, cyclohexane butyrate, and the mixtures thereof, and
(b) at least one aminophenol.

2. The catalyst system according to claim 1, wherein component (a) is selected from the group consisting of zinc stearate, cobalt stearate, zinc glycinate, zinc naphthenate, and mixtures thereof.

3. The catalyst system according to claim 1, wherein the aminophenol is 2-Aminophenol.

4. The catalyst system according to claim 1, wherein it represents the combination of (a) zinc stearate, cobalt stearate, zinc glycinate, zinc naphthenate, and mixtures thereof, and (b) 2-Aminophenol.

5. The catalyst system according to claim 1, wherein it is suitable both for the rearrangement of epoxides in allylic alcohols and production of alpha-beta-unsaturated carbonyl compounds.

6. A method for the rearrangement of epoxides in allylic alcohols, comprising the following steps:
(a) Providing a starting compound having at least one epoxide group;
(b) Providing a catalyst system according to claim 1;
(c) Adding the catalyst system to the starting compound, and heating the mixture to temperatures within the range of from about 155 to about 250° C. over a period of from about 1 to about 10 hours; and
(d) Allowing the mixture to cool, and, optionally, separation of the one or more allylic alcohols formed from the residue.

7. A method for the rearrangement of epoxides and Oppenauer oxidation, in alpha,beta-unsaturated carbonyl compounds, comprising the following steps:
(a) Providing a starting compound having at least one epoxide group;
(b) Providing a catalyst system according to claim 1;
(c) Providing a consumption agent;
(d) Adding the catalyst system to the starting compound and the consumption agent ketone, and heating the mixture to temperatures within the range of from about 170 to about 250° C. over a period of from about 1 to about 10 hours; and
(e) Allowing the mixture to cool and, optionally, separation of the one or more alpha-beta-unsaturated carbonyl compounds formed from the residue.

8. The method according to claim 6, wherein the one or more reactions are performed at temperatures within the range of from about 200 to about 230° C.

9. The method according to claim 6, wherein catalyst component (a) is employed in amounts of from about 0.05 to about 5 mol %, and catalyst component (b) is employed in amounts of from about 0.01 to about 0.00001 mol % each based on the starting compounds.

10. The method according to claim 6, wherein the one or more reactions are carried out in the presence of a solvent.

11. The method according to claim 7, wherein the rearrangement and the Oppenauer oxidation are performed simultaneously as a one-pot reaction, or subsequently.

12. The method according to claim 7, wherein the consumption agent is added only in the course of reaction.

13. The method according to claim 7, wherein an aldehyde, a ketone, or a quinone is used as a consumption agent.

14. The method according to claim 7, wherein the alcohol formed from the consumption agent is periodically removed from the reaction mixture.

15. The method according to claim 6, wherein 1,2-Limonene epoxide is used as a starting compound.

16. A pre-formed catalyst, comprising at least one of the following structures (1) to (8): [chemical structure]
